(19) United States
(12) Patent Application Publication
(10) Pub. No.: US 2003/0198643 A1
(43) Pub. Date: Oct. 23, 2003

(54) Adjuvant enhanced immunotherapy

(76) Inventor: Yingjuan Lu, West Lafayette, IN (US)

Correspondence Address: BARNES & THORNBURG, 11 SOUTH MERIDIAN, INDIANAPOLIS, IN, 46204

(21) Appl. No.: 10/417,903

(22) Filed: Apr. 17, 2003

Related U.S. Application Data
(60) Provisional application No. 60/373,818, filed on Apr. 19, 2002.

(51) Int. Cl. A61K 38/19; A61K 31/704; A61K 39/00

(52) U.S. Cl. 424/185.1; 514/33; 424/85.1

(57) ABSTRACT

An improved method is provided for treating disease states characterized by the existence of pathogenic cell populations. In accordance with the improved method, cell-targeted ligand-immunogen or ligand-hapten complexes are administered to a diseased host to redirect the host immune response to the pathogenic cells which have an accessible binding site for the ligand. The method comprises the step of administering to the host a ligand-immunogen or ligand-hapten conjugate composition comprising a complex of the ligand and the immunogen or hapten wherein the immunogen/hapten is recognized by an endogenous antibody in the host or directly by an immune cell in the host. The improvement to the method comprises the step of using a T_{H}1-biasing adjuvant to enhance the immune response to cell-bound ligand-immunogen or ligand-hapten conjugates.

### CROSS REFERENCE TO RELATED APPLICATIONS

This application claims priority under 35 U.S.C. §119(e) to U.S. Provisional Application Serial No. 60/373,818, filed on Apr. 19, 2002.

### FIELD OF THE INVENTION

The invention relates to an improved method for treating disease states characterized by the existence of pathogenic cell populations. More particularly, cell-targeted ligand-immunogen or ligand-hapten conjugates are administered to a diseased host to direct the host immune response to the pathogenic cells. The improvement to the method comprises using an adjuvant that biases the immune response towards a T_{H}1 response to enhance the immune response to the immunogen.

### BACKGROUND OF THE INVENTION

The mammalian immune system provides a means for the recognition and elimination of tumor cells, other pathogenic cells, and invading foreign pathogens. While the immune system normally provides a strong line of defense, there are still many instances where cancer cells, other pathogenic cells, or infectious agents evade a host immune response and proliferate or persist with concomitant host pathogenicity. Chemotherapeutic agents and radiation therapies have been developed to eliminate replicating neoplasms. However, most, if not all, of the currently available chemotherapeutic agents and radiation therapy regimens have adverse side effects because they work not only to destroy cancer cells, but they also affect normal host cells, such as cells of the hematopoietic system. Furthermore, chemotherapeutic agents have limited efficacy in instances where host drug resistance is developed.

Foreign pathogens can also proliferate in a host by evading a competent immune response or where the host immune system has been compromised by drug therapies or by other health problems. Although many therapeutic compounds have been developed, many pathogens are or have become resistant to such therapeutics. The capacity of cancer cells and infectious organisms to develop resistance to therapeutic agents, and the adverse side effects of the currently available anticancer drugs, highlight the need for the development of new therapies specific for pathogenic cell populations and with reduced host toxicity.

Researchers have developed therapeutic protocols for destroying cancer cells by targeting cytotoxic compounds specifically to such cells. These protocols utilize toxins conjugated to ligands that bind to receptors unique to or overexpressed by cancer cells in an attempt to minimize delivery of the toxin to normal cells. Using this approach certain immunotoxins have been developed consisting of antibodies directed to specific receptors on pathogenic cells, the antibodies being linked to toxins such as ricin, Pseudomonas exotoxin, Diptheria toxin, and tumor necrosis factor. These immunotoxins target tumor cells bearing the specific receptors recognized by the antibody (Olsnes, S., Immunol. Today, 10, pp. 291-295, 1989; Melby, E. L., Cancer Res., 53(8), pp. 1755-1760, 1993; Better, M. D., PCT Publication Number WO 91/07418, published May 30, 1991).

Another approach for selectively targeting populations of cancer cells or foreign pathogens in a host is to enhance the host immune response against the pathogenic cells, thereby avoiding the need for administration of compounds that may also exhibit independent host toxicity. One reported strategy for immunotherapy is to bind antibodies, for example, genetically engineered multimeric antibodies, to the tumor cell surface to display the constant region of the antibodies on the cell surface and thereby induce tumor cell killing by various immune-system mediated processes. (De Vita, V. T., Biologic Therapy of Cancer, 2d ed. Philadelphia, Lippincott, 1995; Soulillou, J. P., U.S. Pat. No. 5,672,486). However, this approach has been complicated by the difficulties in defining tumor-specific antigens. Another approach to relying on host immune competency is the targeting of an anti-T cell receptor antibody or anti-Fc receptor antibody to tumor cell surfaces to promote direct binding of immune cells to tumors (Kranz, D. M., U.S. Pat. No. 5,547,668). A vaccine-based approach has also been described which relies on a vaccine comprising antigens fused to cytokines, with the cytokine modifying the immunogenicity of the vaccine antigen, and, thus, stimulating the immune response to the pathogenic agent (Pillai, S., PCT Publication Number WO 91/11146, published Feb. 7, 1991). That method relies on indirect modulation of the immune response reported. Another approach for killing unwanted cell populations utilizes IL-2 or Fab fragments of anti-thymocyte globulin linked to antigens to eliminate unwanted T cells; however, based on reported experimental data, the method appears to eliminate only 50% of the targeted cell population, and results in nonspecific cell killing in vivo (i.e., 50% of peripheral blood lymphocytes that are not T cells are also killed (Pouletty, P., PCT publication number WO 97/37690, published Oct. 16, 1997)). Thus, there remains a significant need for therapies directed to treatment of disease states characterized by the existence of pathogenic cell populations in an affected host.

The immune system may exhibit both specific and nonspecific immunity with specific immunity being mediated by B and T lymphocytes which display receptors on their surfaces for specific antigens. The specific immune response may involve humoral immunity (i.e., B cell activation with the production of antibodies), and cell-mediated immunity (i.e., activation of T cells, such as cytotoxic T lymphocytes, helper T lymphocytes, including T_{H}1 and T_{H}2 cells, and antigen-presenting cells). T_{H}1 responses elicit complement fixing antibodies, activation of cytotoxic T lymphocytes, and strong delayed-type hypersensitivity reactions and are associated with the production of IL-2, IL-12, TNF, lymphotoxin, and γ-interferon. T_{H}2 responses are associated with the production of IgE, and IL-4, IL-5, IL-6, and IL-10. A specific immune response involves not only specificity, but also memory so that immune cells previously exposed to an antigen can rapidly respond to that same antigen upon future exposure to the antigen.

Adjuvants are compounds or materials that stimulate immune responses, for example, by augmenting the immunogenicity of an antigen, either when administered with the antigen or when administered prior to the antigen. Adjuvants can act either nonspecifically, stimulating the immune response to a wide variety of antigens, or specifically (i.e., stimulating the immune response in an antigen-specific manner). Adjuvants that enhance specific immunity can act by stimulating the cell-mediated immune response or the humoral response or both. Adjuvants that stimulate the cell-mediated immune response can bias the immune response towards a T_{H}1 or a T_{H}2 response. Adjuvants that stimulate the humoral immune response can induce the production of an antibody isotype profile that differs depending on the adjuvant used. In this regard, different adjuvants can stimulate the production of 1.) different antibody isotypes, 2.) different levels of antibodies of each isotype, and 3.) can stimulate the production of antibodies with differing affinities, resulting in divergent antibody populations depending on the adjuvant used.

Saponins are glycosidic compounds that are widely distributed among higher plants and in some marine invertebrates of the phylum Echinodermata (ApSimon et al., Stud. Org. Chem. 17:273-286 (1984)). Saponins consist of an aglycone attached to one or more linear or branched sugar chains, and have molecular weights ranging from 600 to 2000 daltons or greater. Saponins are known to exhibit adjuvant activity.

The quillajasaponins are a family of closely related O-acylated triterpene glycoside structures, and are isolated from the bark of the Quillaja saponaria Molina tree. Quillajasaponins are functionally well-characterized and are known to exhibit adjuvant activity. The quillajasaponins stimulate both the cell-mediated and humoral immune responses. An aldehyde group on the triterpenoid group of quillajasaponins is responsible for inducing cell-mediated immunity, and carbohydrate moieties on the quillajasaponins appear to enhance humoral immunity. The quillajasaponins generally induce a strong T_{H}1 response.

### SUMMARY OF THE INVENTION

An improvement is provided to a method of eliminating pathogenic cell populations in a host. The method is based on increasing host immune system recognition of and response to pathogenic cell populations by increasing the antigenicity of the pathogenic cells to enhance an endogenous immune response-mediated elimination of the population of pathogenic cells. In accordance with the method, ligand-immunogen or ligand-hapten conjugates are administered to the host for binding to the surface of the tumor cells or pathogenic organisms and the conjugates “label” the cells of the targeted cell population with the immunogen or hapten, thereby triggering an immune-mediated response directed at the labeled cell population. Antibodies existing or produced in the host bind to the immunogen or hapten and trigger endogenous immune responses. Alternatively, the immunogen or hapten can be recognized directly by immune cells in the host. The improvement to the method comprises using a T_{H}1-biasing adjuvant to enhance the immune response to the immunogen/hapten.

The method comprises administration of a ligand-immunogen conjugate or a ligand-hapten conjugate wherein the ligand is capable of specific binding to a population of pathogenic cells in vivo, and the ligand conjugated immunogen/hapten is capable of being recognized by antibodies or directly by immune cells in the host. The immune system-mediated elimination of the pathogenic cells is directed by the binding of the immunogen/hapten conjugated ligand to a receptor, a transporter, or other surface-presented protein uniquely expressed, overexpressed, or preferentially expressed by the pathogenic cell. A surface-presented protein uniquely expressed, overexpressed, or preferentially expressed by the pathogenic cell is a receptor not present or present at lower amounts on non-pathogenic cells providing a means for selective elimination of the pathogenic cells.

The targeted pathogenic cell population can be a cancer cell population, virus-infected endogenous cells, or a population of exogenous organisms such as bacteria, mycoplasma yeast or fungi. Antibody binding to the cell-bound ligand-immunogen or ligand-hapten conjugate results in complement-mediated cytotoxicity, antibody-dependent cell-mediated cytotoxicity, antibody opsonization and phagocytosis, antibody-induced receptor clustering signaling cell death or quiescence or any other humoral or cellular immune response stimulated by antibody binding to cell-bound ligand-immunogen or ligand-hapten conjugates. The immune response can also involve direct recognition of the immunogen/hapten by host immune cells.

At least one additional therapeutic factor, for example, an immune system stimulant, a cell killing agent, a tumor penetration enhancer, a chemotherapeutic agent, a cytotoxic immune cell, or an antimicrobial agent can be administered to the host animal to enhance therapeutic efficiency. In one embodiment, the cytotoxic immune cell is a cytotoxic immune cell population that is isolated, expanded ex vivo, and is then injected into a host animal. In another embodiment an immune stimulant is used and the immune stimulant can be an interleukin such as IL-2, IL-12, or IL-15 or an IFN such as IFN-α, IFN-β, or IFN-9γ, or GM-CSF. In another embodiment the immune stimulant can be a cytokine composition comprising combinations of cytokines, such as IL-2, IL-12 or IL-15 in combination with IFN-α, IFN-β, IFN-γ, or GM-CSF, or any effective combination thereof, or any other effective combination of cytokines.

Thus, in one embodiment a method is provided of enhancing an endogenous immune response-mediated specific elimination of a population of pathogenic cells in a preimmunized host animal harboring the population wherein the members of the cell population have an accessible binding site for a ligand. The method comprises the step of administering to the host a composition comprising an immunogen or a hapten conjugated to the ligand wherein the immunogen or the hapten is recognized by an endogenous antibody in the host or is recognized directly by an immune cell in the host, the improvement comprising the step of preimmunizing the host with the immunogen or an immunogenic hapten-carrier conjugate and a T_{H}1-biasing adjuvant to elicit a preexisting immunity.

In another embodiment, a method is provided of enhancing an immune response in a host animal harboring a population of pathogenic cells to eliminate said pathogenic cell population wherein the pathogenic cells have an accessible binding site for a ligand. The method comprises the steps of administering to the host a T_{H}1-biasing adjuvant, and administering to the host a composition comprising an immunogen conjugated to the ligand.

In another embodiment a composition is provided comprising therapeutically effective amounts of a T_{H}1-biasing adjuvant and a hapten-carrier conjugate wherein the hapten is selected from the group consisting of fluorescein and dinitrophenyl.

In yet another embodiment a composition is provided comprising therapeutically effective amounts of a T_{H}1-biasing adjuvant and a ligand-immunogen conjugate.

In still another embodiment a kit is provided comprising a T_{H}1-biasing adjuvant and a hapten-carrier conjugate wherein the hapten is selected from the group consisting of fluorescein and dinitrophenyl.

In another embodiment a kit is provided comprising a T_{H}1-biasing adjuvant, a hapten-carrier conjugate, and a ligand-hapten conjugate. Alternatively, the kit can comprise a T_{H}1-biasing adjuvant and a ligand-immunogen conjugate, or can further comprise an immunogen.

### BRIEF DESCRIPTION OF THE DRAWINGS

FIG. 1 shows the anti-FITC total IgG and anti-FITC IgG2a responses in mice immunized with KLH-FITC formulated with a saponin adjuvant (i.e., GPI-0100).

FIG. 2 shows the percentage survival of mice, having established intraperitoneal L1210A leukemia, immunized with KLH-FITC/saponin adjuvant and subsequently injected with PBS (control), IL2+IFN-α, or folate-FITC+IL2+IFN-α.

FIG. 3 shows the percentage survival of mice, bearing established intraperitoneal M109 tumors, immunized with KLH-FITC/saponin adjuvant and subsequently injected with PBS, IL2+IFN-α, or folate-FITC+IL2+IFN-α.

FIG. 4 shows the percentage survival of mice, bearing early-stage intraperitoneal M109 tumors, immunized with KLH-FITC/saponin adjuvant and subsequently injected with PBS or folate-FITC.

FIG. 5 shows the percentage survival of mice, bearing established intraperitoneal M109 tumors, immunized with KLH-FITC/saponin adjuvant and subsequently injected with PBS or folate-FITC.

FIG. 6 shows the tumor volume of subcutaneous M109 tumors in mice immunized with KLH-FITC/saponin adjuvant and subsequently injected with PBS, IL2+IFN-α, or folate-FITC+IL2+IFN-α.

FIG. 7 shows the structure of folate-FITC (EC17).

FIG. 8 shows the structure of KLH-FITC (EC90).

### DETAILED DESCRIPTION OF THE INVENTION

An improvement is provided to a method of eliminating pathogenic cell populations in a host. The method is based on increasing host immune system recognition of and response to pathogenic cell populations by increasing the antigenicity of the pathogenic cells to enhance an endogenous immune response-mediated elimination of the population of pathogenic cells. In accordance with the method, ligand-immunogen or ligand-hapten conjugates are administered to the host for binding to the surface of the tumor cells or pathogenic organisms and the conjugates “label” the cells of the targeted cell population with the immunogen or hapten, thereby triggering an immune-mediated response directed at the labeled cell population. Antibodies existing or produced in the host or immune cells in the host bind to the immunogen/hapten and trigger endogenous immune responses. The improvement to the method in accordance with the present invention comprises using a T_{H}1-biasing adjuvant to enhance the immune response to the immunogen/hapten.

The improved method is utilized to enhance an endogenous immune response-mediated elimination of a population of pathogenic cells in a host animal harboring the population of pathogenic cells. The invention is applicable to populations of pathogenic cells that cause a variety of pathologies such as cancer and infectious diseases. Thus, the population of pathogenic cells can be a cancer cell population that is tumorigenic, including benign tumors and malignant tumors, or it can be non-tumorigenic. The cancer cell population can arise spontaneously or by such processes as mutations present in the germline of the host animal or somatic mutations, or it may be chemically-, virally-, or radiation-induced. The invention can be utilized to treat such cancers as carcinomas, sarcomas, lymphomas, Hodgkin's disease, melanomas, mesotheliomas, Burkitt's lymphoma, nasopharyngeal carcinomas, leukemias, and myelomas. The cancer cell population can include, but is not limited to, oral, thyroid, endocrine, skin, gastric, esophageal, laryngeal, pancreatic, colon, bladder, bone, ovarian, cervical, uterine, breast, testicular, prostate, rectal, kidney, liver, and lung cancers.

The population of pathogenic cells can also be an exogenous pathogen or a cell population harboring an exogenous pathogen, e.g., a virus. The present invention is applicable to such exogenous pathogens as bacteria, fungi, viruses, mycoplasma, and parasites. Infectious agents that can be treated with the present invention are any art-recognized infectious organisms that cause pathogenesis in an animal, including such organisms as bacteria that are gram-negative or gram-positive cocci or bacilli, DNA and RNA viruses, including, but not limited to, DNA viruses such as papilloma viruses, parvoviruses, adenoviruses, herpesviruses and vaccinia viruses, and RNA viruses, such as arenaviruses, coronaviruses, rhinoviruses, respiratory syncytial viruses, influenza viruses, picomaviruses, paramyxoviruses, reoviruses, retroviruses, and rhabdoviruses. Of particular interest are bacteria that are resistant to antibiotics such as antibiotic-resistant Streptococcus species and Staphlococcus species, or bacteria that are susceptible to antibiotics, but cause recurrent infections treated with antibiotics so that resistant organisms eventually develop. Such organisms can be treated with the ligand-immunogen or ligand-hapten conjugates of the present invention in combination with lower doses of antibiotics than would normally be administered to a patient to avoid the development of these antibiotic-resistant bacterial strains. The present invention is also applicable to any fungi, mycoplasma species, parasites, or other infectious organisms that cause disease in animals. Examples of fungi that can be treated with the method of the present invention include fungi that grow as molds or are yeastlike, including, for example, fungi that cause diseases such as ringworm, histoplasmosis, blastomycosis, aspergillosis, cryptococcosis, sporotrichosis, coccidioidomycosis, paracoccidio-idomycosis, and candidiasis. The present invention can be utilized to treat parasitic infections including, but not limited to, infections caused by somatic tapeworms, blood flukes, tissue roundworms, ameba, and Plasmodium, Trypanosoma, Leishmania, and Toxoplasma species. Parasites of particular interest are those that express folate receptors and bind folate; however, the literature is replete with reference to ligands exhibiting high affinity for infectious organisms. For example, penicillins and cephalosporins known for their antibiotic activity and specific binding to bacterial cell wall precursors can similarly be used as ligands for preparing ligand-immunogen or ligand-hapten conjugates for use in accordance with this invention. The ligand-immunogen or ligand-hapten conjugates of the invention can also be directed to a cell population harboring endogenous pathogens wherein pathogen-specific antigens are preferentially expressed on the surface of cells harboring the pathogens, and act as receptors for the ligand with the ligand specifically binding to the antigen.

The method of the present invention can be used for both human clinical medicine and veterinary applications. Thus, the host animals harboring the population of pathogenic organisms and treated with ligand-immunogen or ligand-hapten conjugates can be humans or, in the case of veterinary applications, may be a laboratory, agricultural, domestic, or wild animal. The present invention can be applied to host animals including, but not limited to, humans, laboratory animals such rodents (e.g., mice, rats, hamsters, etc.), rabbits, monkeys, chimpanzees, domestic animals such as dogs, cats, and rabbits, agricultural animals such as cows, horses, pigs, sheep, goats, and wild animals in captivity such as bears, pandas, lions, tigers, leopards, elephants, zebras, giraffes, gorillas, dolphins, and whales.

In one embodiment of the improved method, the host is preimmunized with an immunogen or a hapten-carrier (e.g., KLH or BSA) conjugate and a T_{H}1-biasing adjuvant to elicit a preexisting immunity to the immunogen or hapten. The ligand-immunogen or ligand-hapten conjugate is then administered to the host resulting in an humoral or cell-mediated immune response, or both, directed against the ligand-immunogen or ligand-hapten conjugate bound to the targeted pathogenic cells.

In another embodiment, the preexisting immunity can be an innate immunity against the immunogen (e.g., an immunogen such as a superantigen or muramyl dipeptide). In this embodiment, the T_{H}1-biasing adjuvant and the ligand-immunogen conjugate can be co-administered to enhance the immune response derived, at least in part, from the innate immunity.

In another embodiment, the preexisting immunity can be an immunity developed via normally scheduled vaccinations or prior natural exposure to an antigen (e.g., poliovirus, tetanus, influenza, and the like). In this embodiment, the immunogen comprises an antigen that elicited the preexisting immunity and the T_{H}1-biasing adjuvant and the ligand-immunogen conjugate are co-administered to enhance the immune response resulting from the preexisting immunity.

In yet another embodiment, the ligand-immunogen conjugate and the T_{H}1-biasing adjuvant can be co-administered to elicit an immune response where there is no preexisting immunity. In this embodiment, the T_{H}1-biasing adjuvant enhances the immune response to the immunogen upon co-administration of the adjuvant and the ligand-immunogen conjugate.

In another embodiment, where there is no preexisting immunity, the ligand-immunogen conjugate, the T_{H}1-biasing adjuvant, and passively administered antibodies can be co-administered. In this embodiment, the passively administered antibodies help to augment the immune response to the immunogen.

For all of the embodiments described herein, “co-administration” is defined as administration at a time prior to, at the same time as, or at a time following administration of the ligand-immunogen, ligand-hapten, or hapten-carrier conjugate or the immunogen. In accordance with the invention, “co-administration” can also mean administration in the same or different solutions.

Adjuvants suitable for use in accordance with the invention are adjuvants that bias the immune response towards a T_{H}1 response. An adjuvant-induced T_{H}1-biased immunity can be measured in mice through immunoglobulin isotype distribution analysis. Adjuvants that bias the immune response towards a T_{H}1 response are adjuvants that preferentially increase IgG2a antibody levels in mice relative to IgG1 antibody levels. An antigen-specific IgG2a/IgG1 ratio of ≧1 can be indicative of a T_{H}1-like antibody subclass pattern. However, in accordance with the invention, any adjuvant that increases the production of antigen-specific antibodies, and, at the same time, increases the relative IgG2a/IgG1 ratio to about ≧0.3 drives the immune response towards a T_{H}1-biased immune response. Such adjuvants can include saponin adjuvants (e.g., the quillajasaponins, including lipid-modified quillajasaponin adjuvants), CpG, 3-deacylated monophosphoryl lipid A (MPL), Bovine Calmette-Guerin (BCG), double stem-loop immunomodulating oligodeoxyribonucleotides (d-SLIM), heat-killed Brucella abortus (HKBA), heat-killed Mycobacterium vaccae (SRL172), inactivated vaccinia virus, cyclophosphamide, prolactin, thalidomide, actimid, revimid, and the like. Saponin adjuvants and methods of their preparation and use are described in detail in U.S. Pat. Nos. 5,057,540, 5,273,965, 5,443,829, 5,508,310, 5,583,112, 5,650,398, 5,977,081, 6,080,725, 6,231,859, and 6,262,029 incorporated herein by reference.

The ligand-immunogen or ligand-hapten conjugates can be selected from a wide variety of ligands, immunogens, and haptens. The ligands should be capable of preferentially targeting a population of pathogenic cells in the host animal due to preferential or overexpression of a receptor for the ligand, accessible for ligand binding, on the pathogenic cells. Acceptable ligands include folic acid, analogs of folic acid and other folate receptor-binding molecules, other vitamins, peptide ligands identified from library screens, tumor-specific peptides, tumor-specific aptamers, tumor-specific carbohydrates, tumor-specific monoclonal or polyclonal antibodies, Fab or scFv (i.e., a single chain variable region) fragments of antibodies such as, for example, an Fab fragment of an antibody directed to EphA2 or other proteins specifically expressed or uniquely accessible on metastatic cancer cells, small organic molecules derived from combinatorial libraries, growth factors, such as EGF, FGF, insulin, and insulin-like growth factors, and homologous polypeptides, somatostatin and its analogs, transferrin, lipoprotein complexes, bile salts, selecting, steroid hormones, Arg-Gly-Asp containing peptides, retinoids, various Galectins, δ-opioid receptor ligands, cholecystokinin A receptor ligands, ligands specific for angiotensin AT1 or AT2 receptors, peroxisome proliferator-activated receptor γ ligands, β-lactam antibiotics, small organic molecules including antimicrobial drugs, and other molecules that bind specifically to a receptor preferentially expressed on the surface of tumor cells or on an infectious organism, or fragments of any of these molecules. Of interest in the case of ligands that bind to infectious organisms, are any molecules, such as antibiotics or other drugs, that are known in the art to preferentially bind to the microorganism. The invention also applies to ligands which are molecules, such as antimicrobial drugs, designed to fit into the binding pocket of a particular receptor, based on the crystal structure of the receptor, or other cell surface protein, and wherein such receptors are preferentially expressed on the surface of tumors, bacteria, viruses, mycoplasma, fungi, parasites, or other pathogens. It is also contemplated, in one embodiment, that ligands binding to any tumor antigens or other molecules preferentially expressed on the surface of tumor cells can be utilized.

In one embodiment the ligand is a vitamin or an analog or derivative thereof. Acceptable vitamins include niacin, pantothenic acid, folic acid, riboflavin, thiamine, biotin, vitamin B₁₂, and the lipid soluble vitamins A, D, E and K. These vitamins, and their receptor-binding analogs and derivatives, constitute the targeting entity that forms the ligand-immunogen or ligand-hapten conjugates for use in accordance with the invention. Preferred vitamin moieties include folic acid, biotin, riboflavin, thiamine, vitamin B₁₂, and receptor-binding analogs and derivatives of these vitamin molecules, and other related vitamin receptor-binding molecules (see U.S. Pat. Nos. 5,108,921, 5,416,016, and 5,635,382 incorporated herein by reference). Exemplary of a vitamin analog is a folate analog containing a glutamic acid residue in the D configuration (folic acid normally contains one glutamic acid in the L configuration linked to pteroic acid).

The binding site for the ligand can include receptors for any molecule capable of specifically binding to a receptor wherein the receptor or other protein is preferentially expressed on the population of pathogenic cells, including, for example, receptors for growth factors, vitamins, peptides, including opioid peptides, hormones, antibodies, carbohydrates, and small organic molecules. The binding site can also be a binding site for any molecule, such as an antibiotic or other drug, where the site is known in the art to preferentially exist on microorganisms. For example, the subject binding sites may be binding sites in the bacterial cell wall for a β-lactam antibiotic such as penicillin, or binding sites for an antiviral agent uniquely present on the surface of a virus. The invention also applies to binding sites for ligands, such as antimicrobial drugs, designed to fit into the binding site of the receptor, based on the crystal structure of the receptor, and wherein the receptor is preferentially expressed on the surface of the pathogenic cells or organisms.

It is also contemplated that tumor-specific antigens can function as binding sites for ligands. An example of a tumor-specific antigen that could function as a binding site for ligand-immunogen or ligand-hapten conjugates is an extracellular epitope of a member of the Ephrin family of proteins, such as EphA2. EphA2 expression is restricted to cell-cell junctions in normal cells, but EphA2 is distributed over the entire cell surface in metastatic tumor cells. Thus, EphA2 on metastatic cells would be accessible for binding to, for example, an Fab fragment of an antibody conjugated to an immunogen or a hapten, whereas the protein would not be accessible for binding to the Fab fragment on normal cells, resulting in a ligand-immunogen or ligand-hapten conjugate specific for metastatic cancer cells. The invention further contemplates the use of combinations of ligand-immunogen or ligand-hapten conjugates to maximize targeting of the pathogenic cells for elimination by the immune response.

Suitable immunogens include antigens or antigenic peptides against which a preexisting immunity has developed via normally scheduled vaccinations or prior natural exposure to such agents as poliovirus, tetanus, typhus, rubella, measles, mumps, pertussis, tuberculosis, and influenza antigens, and α-galactosyl groups. In such cases, the ligand-immunogen conjugates are used to redirect a previously acquired humoral or cellular immunity to a population of pathogenic cells in the host animal for elimination of the foreign cells or pathogenic organisms, and the T_{H}1-biasing adjuvant augments the immune response to enhance the elimination of the pathogenic cells.

Antigens or antigenic peptides to which the host animal has developed an innate immunity (e.g., superantigens and muramyl dipeptide) are also suitable immunogens for use in accordance with the invention. In this embodiment the T_{H}1-biasing adjuvant and the ligand-immunogen conjugates are co-administered and the adjuvant enhances the immune response to the immunogen resulting from innate immunity.

In cases where a preexisting immunity does not exist, a preexisting immunity can be developed by preimmunization with an immunogen or a hapten. In such cases a novel preexisting immunity can be developed through immunization with the immunogen or hapten (e.g., fluorescein, dinitrophenyl, trinitrophenyl, α-gal epitopes, synthetic peptides or glycopeptides derived from common viruses, bacteria, carbohydrates, oligosaccharides, gangliosides, and low molecular weight drugs). In embodiments where a hapten is used, the hapten is typically conjugated to a carrier to form a hapten-carrier conjugate. The host is preimmunized with the hapten-carrier conjugate and the T_{H}1-biasing adjuvant. The T_{H}1-biasing adjuvant enhances the immune response to the hapten upon subsequent administration of the ligand-hapten conjugate. In embodiments where the immunogen is not a hapten, a preexisting immunity can be developed by preimmunization with the immunogen and the T_{H}1-biasing adjuvant.

In embodiments where a preexisting immunity does not exist, any immunogen that induces an immune response upon co-administration of the T_{H}1-biasing adjuvant and the ligand-immunogen conjugate can be used.

Carriers that can be used in accordance with the invention include keyhole limpet hemocyanin (KLH), haliotis tuberculata hemocyanin (HtH), inactivated diptheria toxin, inactivated tetanus toxoid, purified protein derivative (PPD) of Mycobacterium tuberculosis, bovine serum albumin (BSA), ovalbumin (OVA), g-globulins, thyroglobulin, peptide antigens, and synthetic carriers, such as poly-L-lysine, dendrimer, and liposomes.

The ligand or the carrier (e.g., KLH or BSA) can be conjugated to the immunogen or the hapten by using any art-recognized method of forming a complex. This can include covalent, ionic, or hydrogen bonding of the carrier or ligand to the immunogen or hapten, either directly or indirectly via a linking group such as a divalent linker. The hapten-carrier, ligand-immunogen, and ligand-hapten conjugates are typically formed by covalent bonding through the formation of amide, ester or imino bonds between acid, aldehyde, hydroxy, amino, or hydrazo groups on the respective components of the conjugates. In embodiments where a linker is used, the linker typically comprises about 1 to about 30 carbon atoms, more typically about 2 to about 20 carbon atoms. Lower molecular weight linkers (i.e., those having an approximate molecular weight of about 20 to about 500) are typically employed. Also, in accordance with this invention the linker can comprise an indirect means for associating the ligand or the carrier with the immunogen or the hapten, such as by connection through intermediary linkers, spacer arms, or bridging molecules. Both direct and indirect means for association should not prevent the binding of the ligand to the receptor on the cell membrane for operation of the method of the present invention.

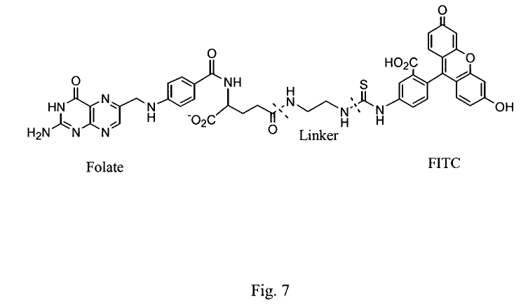

In one embodiment, the ligand is folic acid, an analog of folic acid, or any other folate-receptor binding molecule, and the folate ligand is conjugated to the immunogen or hapten by a procedure that utilizes trifluoroacetic anhydride to prepare γ-esters of folic acid via a pteroyl azide intermediate. This procedure results in the synthesis of a folate ligand, conjugated to the immunogen or hapten only through the γ-carboxy group of the glutamic acid groups of folate (see FIG. 7) wherein the γ-conjugate binds to the folate receptor with high affinity, avoiding the formation of mixtures of an α-conjugate and the γ-conjugate. Alternatively, pure α-conjugates can be prepared from intermediates wherein the γ-carboxy group is selectively blocked, the α-conjugate is formed and the γ-carboxy group is subsequently deblocked using art-recognized organic synthesis protocols and procedures.

The endogenous immune response-mediated elimination of the pathogenic cell population is enhanced by immunization with the T_{H}1-biasing adjuvant. The endogenous immune response can include an humoral response, a cell-mediated immune response, and any other immune response endogenous to the host animal, including complement-mediated cell lysis, antibody-dependent cell-mediated cytoxicity (ADCC), antibody opsonization leading to phagocytosis, clustering of receptors upon antibody binding resulting in signaling of apoptosis, antiproliferation, or differentiation, and direct immune cell recognition of the delivered immunogen/hapten. It is also contemplated that the endogenous immune response will employ the secretion of cytokines that regulate such processes as the multiplication and migration of immune cells. The endogenous immune response can include the participation of such immune cell types as B cells, T cells, including helper and cytotoxic T cells, macrophages, natural killer cells, neutrophils, LAK cells, and the like.

It is contemplated that the preexisting antibodies, induced antibodies, or passively administered antibodies will be redirected to the tumor cells or infectious organisms by preferential binding of the ligand-immunogen or ligand-hapten conjugates to these invading cells or organisms and that the pathogenic cells will be killed by the immune responses described above. The cytotoxic process can also involve secondary responses that arise when the attracted antigen-presenting cells phagocytose the unwanted cells and present natural tumor antigens or antigens of foreign pathogens to the cellular arm of the immune system for elimination of the cells or organisms bearing the antigens.

As discussed above, the immune response can be induced by such processes as normally scheduled vaccination, or active immunization with a natural immunogen or an unnatural immunogen or hapten (e.g., fluorescein or dinitrophenyl), with the unnatural immunogen or hapten inducing a novel immunity. Active immunization can involve multiple injections of the natural immunogen or unnatural immunogen or hapten (e.g., as a hapten-carrier conjugate) scheduled outside of a normal vaccination regimen to induce immunity. The T_{H}1-biasing adjuvant can be administered with the immunogen or hapten using any immunization schedule, such as at a time prior to, at the same time as, or at a time following administration of a natural or an unnatural immunogen or hapten. The T_{H}1-biasing adjuvant can be administered in the same solution or in a different solution than the immunogen or hapten. The immune response can also result from an innate immunity where the host animal has a natural preexisting immunity, such as an immunity to α-galactosyl groups, and, in the case of an innate immunity, the T_{H}1-biasing adjuvant augments the immune response resulting from the innate immunity.

At least one additional composition comprising a therapeutic factor can be administered to the host in combination with the above-detailed methodology, to enhance the endogenous immune response-mediated elimination of the population of pathogenic cells, or more than one additional therapeutic factor can be administered. The therapeutic factor can be selected from a compound capable of stimulating an endogenous immune response, a chemotherapeutic agent, an antimicrobial agent, or other therapeutic factor capable of complementing the efficacy of the administered ligand-immunogen or ligand-hapten conjugate, such as a cytoxic immune cell. In one embodiment, the cytotoxic immune cell is a cytotoxic immune cell population that is isolated, expanded ex vivo, and is then injected into a host animal. The method of the invention can also be performed by administering to the host, in addition to the above-described conjugates, compounds or compositions capable of stimulating an endogenous immune response including, but not limited to, cytokines or immune cell growth factors such as interleukins 1-18, IL-23, stem cell factor, basic FGF, EGF, G-CSF, GM-CSF, FLK-2 ligand, FLT-3 ligand, HILDA, MIP-1α, TGF-α, TGF-β, M-CSF, IFN-α, IFN-β, IFN-γ, soluble CD23, LIF, and combinations thereof.

Therapeutically effective combinations of these cytokines can also be used. In one embodiment, for example, therapeutically effective amounts of IL-2, for example, in amounts ranging from about 0.1 MIU/m²/dose/day to about 60 MIU/m²/dose/day in a multiple dose daily regimen, and IFN-α, for example, in amounts ranging from about 0.1 MIU/m²/dose/day to about 10 MIU/m²/dose/day in a multiple dose daily regimen, can be used (MIU=million international units; m²=approximate body surface area of an average human). In another embodiment IL-12 and IFN-α are used in therapeutically effective amounts, and in yet another embodiment IL-15 and IFN-α are used in therapeutically effective amounts. In an alternate embodiment, IL-2, IFN-α or IFN-γ, and GM-CSF are used in combination. The therapeutic factor(s) used, such as IL-2, IL-12, IL-15, IFN-α, IFN-γ, and GM-CSF, including combinations thereof, can activate natural killer cells and/or T cells. Alternatively, the therapeutic factor or combinations thereof, including an interleukin in combination with an interferon and GM-CSF, can activate other immune effector cells such as macrophages, B cells, neutrophils, NK cells, NKT cells, T cells, LAK cells, or the like. The invention also contemplates the use of any other effective combination of cytokines including combinations of other interleukins and interferons and colony stimulating factors.

Chemotherapeutic agents, which are cytotoxic themselves and can work to enhance tumor permeability, suitable for use as therapeutic factors in accordance with the invention include adrenocorticoids, alkylating agents, antiandrogens, antiestrogens, androgens, estrogens, antimetabolites such as cytosine arabinoside, purine analogs, pyrimidine analogs, and methotrexate, busulfan, carboplatin, chlorambucil, cisplatin and other platinum compounds, tamoxiphen, taxol, cyclophosphamide, plant alkaloids, prednisone, hydroxyurea, teniposide, antibiotics such as mitomycin C and bleomycin, nitrogen mustards, nitrosureas, vincristine, vinblastine, inflammatory and proinflammatory agents, and any other art-recognized chemotherapeutic agent. Other therapeutic factors that can be administered with the present conjugates, include penicillins, cephalosporins, vancomycin, erythromycin, clindamycin, rifampin, chloramphenicol, aminoglycosides, gentamicin, amphotericin B, acyclovir, trifluridine, ganciclovir, zidovudine, amantadine, ribavirin, and any other art-recognized antimicrobial compound.

The therapeutic factor can also be an antibody directed against the immunogen or hapten, such as natural antibodies collected from serum or monoclonal antibodies that may or may not be genetically engineered antibodies, including humanized antibodies, and can be passively administered to the host animal to augment the elimination of the pathogenic cells. The passively administered antibodies can be co-administered with the ligand-immunogen or ligand-hapten conjugate.

The elimination of the population of pathogenic cells will comprise a reduction or elimination of tumor mass or of pathogenic organisms resulting in a therapeutic response. Thus, in accordance with the invention “elimination” of pathogenic cells means a partial or complete elimination of the cells. In the case of a tumor, the elimination can be an elimination of cells of the primary tumor or of cells that have metastasized or are in the process of dissociating from the primary tumor. A prophylactic treatment to prevent return of a tumor after its removal by any therapeutic approach including surgical removal of the tumor, radiation therapy, chemotherapy, or biological therapy is also contemplated in accordance with this invention and is considered to be an elimination of pathogenic cells. The prophylactic treatment can be an initial treatment with the T_{H}1-biasing adjuvant and the hapten-carrier conjugate or the immunogen followed by treatment with the ligand-immunogen or ligand-hapten conjugate, such as treatment in a multiple dose daily regimen, and/or can be an additional treatment or series of treatments with the ligand-immunogen or ligand-hapten conjugate after an interval of days or months following the initial treatments(s) with or without administration of the T_{H}1-biasing adjuvant.

The invention is also directed to a composition comprising therapeutically effective amounts of a T_{H}1-biasing adjuvant and a hapten-carrier conjugate. In this embodiment the hapten can be fluorescein or dinitrophenyl or any other hapten. In another embodiment a composition is provided comprising therapeutically effective amounts of a T_{H}1-biasing adjuvant and a ligand-immunogen conjugate. This composition can further comprise an amount of the therapeutic factor effective to enhance the elimination of the pathogenic cells. The therapeutic factor is selected from the group consisting of a cell killing agent, a tumor penetration enhancer, a chemotherapeutic agent, an antimicrobial agent, a cytotoxic immune cell, and a compound capable of stimulating an endogenous immune response. In the embodiment where the therapeutic factor is a compound capable of stimulating an endogenous immune response, the therapeutic factor can comprise a cytokine such as IL-2, IL-12, IL-15, or IL-23 or combinations of cytokines, including IL-2, IL-12, IL-15, or IL-23 and interferons such as IFN-α, IFN-β, and IFN-γ and combinations of interferons, interleukins, and colony stimulating factors, such as GM-CSF. Kits comprising the above-described components are also contemplated. A kit comprising a T_{H}1-biasing adjuvant, a hapten-carrier conjugate, and a ligand-hapten conjugate is also contemplated. In another embodiment the kit can comprise an immunogen, a T_{H}1-biasing adjuvant, and a ligand-immunogen conjugate. The kits can further comprise a therapeutic factor.

The dosages of the adjuvant, the immunogen, the hapten-carrier conjugate, the ligand-immunogen conjugate, and the ligand-hapten conjugate can vary depending on the host condition, the disease state being treated, the molecular weight of the conjugate or immunogen, route of administration and tissue distribution, and the possibility of co-usage of other therapeutic treatments such as radiation therapy. The effective amounts to be administered to a patient are based on body surface area, patient weight, and physician assessment of patient condition. Effective doses of the adjuvant can range from about 0.01 μg to about 100 mg per patient, or from about 100 μg to about 50 mg per patient, or from about 500 μg to about 10 mg per patient. Effective doses of the hapten-carrier conjugate or the immunogen can range from about 1 μg to about 100 mg per patient, or from about 10 μg to about 50 mg per patient, or from about 50 μg to about 10 mg per patient. Effective doses of the ligand-immunogen or ligand-hapten conjugate can range from about 1 ng/kg to about 1 mg/kg, or from about 1 μg/kg to about 500 μg/kg, or from about 1 μg/kg to about 100 μg/kg.

Any effective regimen for administering the T_{H}1-biasing adjuvant, the immunogen, the hapten-carrier conjugate, the ligand-immunogen conjugate, the ligand-hapten conjugate and the therapeutic factor to redirect the immune response to the tumor cells or infectious organisms can be used. For example, the T_{H}1-biasing adjuvant, the immunogen, the conjugates, and the therapeutic factor can be administered as single doses, or they can be divided and administered as a multiple-dose daily regimen. Further, a staggered regimen, for example, one to three days per week can be used as an alternative to daily treatment, and for the purpose of defining this invention such intermittent or staggered daily regimen is considered to be equivalent to every day treatment and within the scope of this invention. For example, in one embodiment of the invention the host is treated with multiple injections of the ligand-hapten conjugate and the therapeutic factor, after three initial doses of the T_{H}1-biasing adjuvant and the hapten-carrier conjugate, to eliminate the population of pathogenic cells. In another embodiment, the host is injected multiple times (e.g., about 2 up to about 50 times) with the ligand-hapten conjugate, for example, at 12-72 hour intervals or at 48-72 hour intervals. Additional injections of the ligand-hapten conjugate can be administered to the patient at an interval of days or months after the initial injections(s) and the additional injections prevent recurrence of disease. Alternatively, the initial injection(s) of the ligand-hapten conjugate may prevent recurrence of disease.

In another embodiment where a preexisting immunity has been developed by preimmunization with the T_{H}1-biasing adjuvant and an immunogen or a hapten-carrier conjugate, the ligand-immunogen conjugate or ligand-hapten conjugate can be subsequently administered with a therapeutic factor. The therapeutic factor can be administered to the host animal prior to, after, or at the same time as the ligand-immunogen conjugate or the ligand-hapten conjugate and the therapeutic factor can be administered as part of the same composition containing the ligand-immunogen conjugate or the ligand-hapten conjugate or as part of a different composition than the conjugate. Any such therapeutic composition containing the therapeutic factor at a therapeutically effective dose can be used in the present invention. In another embodiment where no preexisting immunity has been developed, the therapeutic factor can be co-administered with the T_{H}1-biasing adjuvant and the ligand-immunogen conjugate.

Additionally, more than one type of immunogen, hapten-carrier conjugate, ligand-immunogen conjugate, or ligand-hapten conjugate can be used. For example, the host animal can be preimmunized with both fluorescein-carrier and dinitrophenyl-carrier conjugates and subsequently treated with fluorescein and dinitrophenyl linked to the same or different ligands in a co-dosing protocol. In the case of chemotherapeutic and antimicrobial agents, the therapeutic factor can be administered at a suboptimal dose along with the ligand-immunogen conjugate or the ligand-hapten conjugate in a combination therapy to avoid development of resistance to the chemotherapeutic or antimicrobial agent by the host animal.

The T_{H}1-biasing adjuvant, the immunogen, the hapten-carrier conjugate, the ligand-immunogen conjugate, the ligand-hapten conjugate and the therapeutic factor are preferably injected parenterally and such injections can be intradermal injections, intraperitoneal injections, subcutaneous injections, intramuscular injections, intravenous injections, or intrathecal injections. Alternatively, the T_{H}1-biasing adjuvant, the immunogen, and the conjugates can be administered to the host animal by other medically useful processes, such as oral administration, and any suitable therapeutic dosage form can be used. Examples of parenteral dosage forms include aqueous solutions of the active agent, in an isotonic saline, 5% glucose or other well-known pharmaceutically acceptable liquid carriers such as liquid alcohols, glycols, esters, and amides. The parenteral dosage form in accordance with this invention can also be in the form of a reconstitutable lyophilizate. In one embodiment, any of a number of prolonged release dosage forms known in the art can be administered such as, for example, the biodegradable carbohydrate matrices described in U.S. Pat. Nos. 4,713,249; 5,266,333; and 5,417,982, the disclosures of which are incorporated herein by reference. In another embodiment a slow pump can be used.

The method of the present invention can be used in combination with additional therapies such as surgical removal of a tumor, radiation therapy, chemotherapy, or biological therapies such as other immunotherapies including, but not limited to, monoclonal antibody therapy, treatment with immunomodulatory agents, adoptive transfer of immune effector cells, treatment with hematopoietic growth factors, cytokines and vaccination.

### EXAMPLE 1

### Curative Effect of Saponin Enhanced Immunotherapy (With Cytokine) in DBA Mice Having Intraperitoneal L1210A Leukemia

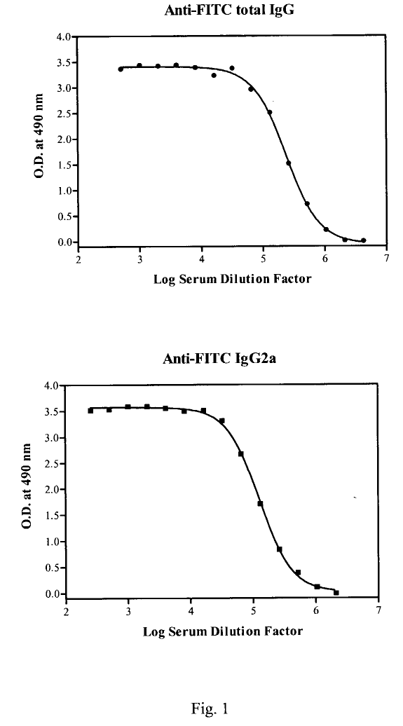

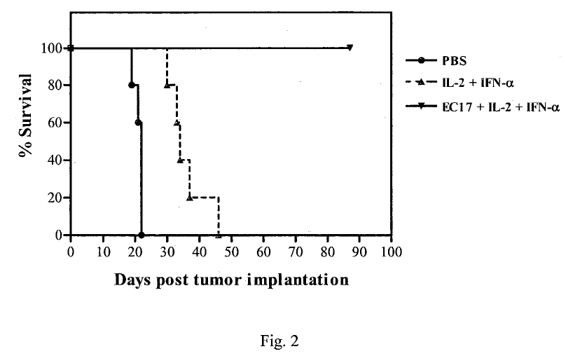

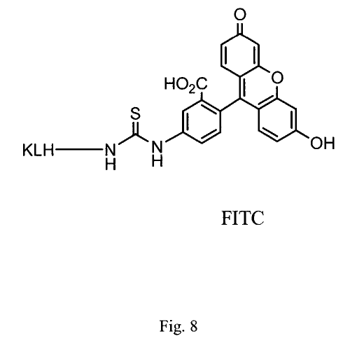

Six to eight week-old (˜20-22 grams) female DBA mice were immunized three times subcutaneously at 2-week intervals with 35 μg of fluorescein isothiocyanate (FITC)-labeled kehole limpet hemocyanin (KLH; see FIG. 8) co-formulated with 100 μg GPI-0100. GPI-0100 is a saponin adjuvant that is a lipid-modified derivative of partially purified quillajasaponins. The preparation and use of GPI-0100 are described in U.S. Pat. No. 6,080,725, incorporated herein by reference. Approximately 1 week after the third immunization, blood samples were collected from treated animals and used in ELISA assays to determine the amount of anti-FITC IgG and IgG2a antibody present (see FIG. 1). After assuring that anti-FITC antibody titers were high in all mice, each animal was injected intraperitoneally approximately 5 weeks after the first immunization with 2.5×10⁴ L1210A cells, a syngeneic mouse leukemia cell line that expresses high levels of the high-affinity folate receptor. The cancer cells were then allowed to proliferate and grow in vivo for 7 days. Thereafter, the tumor-bearing mice were treated intraperitoneally with phosphate buffered saline (PBS) or were co-injected with PBS, IL-2 (250,000 IU/dose), and IFN-α (75,000 IU/dose), or with a folate-FITC conjugate (EC 17; see FIG. 7; 1800 nmol/kg), IL-2 (250,000 IU/dose), and IFN-α (75,000 IU/dose) on days 7, 8, 9, 11, and 14 after tumor cell implantation. Animal gross morphology, behavior, and survival were monitored daily. As shown in FIG. 2, while cytokines alone extended the survival of tumor bearing mice to some degree, the mice treated with EC 17, IL-2, and IFN-α were cured (confirmed by histopathological analysis).

### EXAMPLE 2

### Saponin Enhanced Immunotherapy (With Cytokines) Extended Survival of Balb/c Mice Injected Intraperitoneally with M109 Tumor Cells

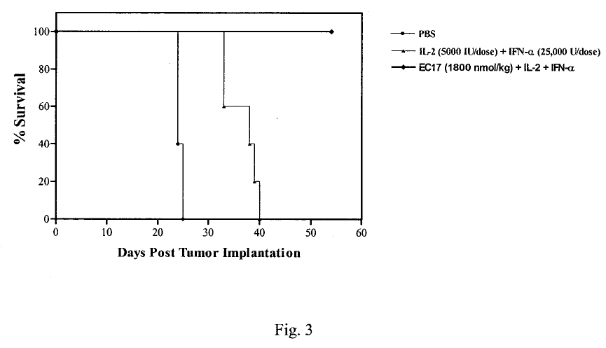

Six to eight week-old (˜20-22 grams) female Balb/c mice were immunized three times subcutaneously at 2-week intervals with 35 μg of KLH-FITC formulated with 100 μg of GPI-0100. After confirming that anti-FITC antibody titers were high in all mice as described in Example 1, each animal was injected intraperitoneally, approximately 5 weeks after the first immunization, with 7.5×10⁵ M109 cells, a syngeneic mouse lung cancer cell line that expresses high levels of the high-affinity folate receptor. The cancer cells were then allowed to proliferate in vivo for 7 days. Thereafter, the tumor-bearing mice were injected subcutaneously with PBS or were co-injected with PBS, IL-2 (5,000 IU/dose), and IFN-α (25,000 IU/dose), or with PBS, EC17 (1800 nmol/kg), IL-2 (5,000 IU/dose), and IFN-α (25,000 IU/dose) on days 7-11, 14-18, and 21-25 after tumor cell implantation. EC17 and IFN-α were dosed at 3 times per week. IL-2 was dosed at 5 times per week. Animal gross morphology, behavior, and survival were monitored daily. As shown in FIG. 3, while cytokines alone extended the survival of tumor bearing mice to some degree, the survival of mice treated with EC 17, IL-2, and IFN-α was prolonged substantially.

### EXAMPLE 3

### Effect of Saponin-Enhanced EC17 Immunotherapy Alone (Without Cytokines) in Balb/c Mice Bearing a One-Day-Old Intraperitoneal M109 Tumor

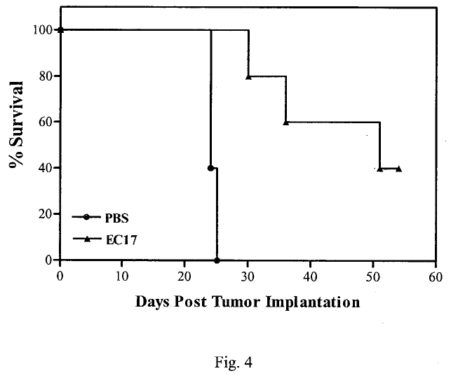

Six to eight week-old (˜20-22 grams) female Balb/c mice were immunized three times subcutaneously at 2-week intervals with 35 μg of KLH-FITC formulated with 100 μg of GPI-0100. After confirming that anti-FITC antibody titers were high in all mice as described in Example 1, each animal was injected intraperitoneally, approximately 5 weeks after the first immunization, with 7.5×10⁵ M109 cells. One day later, the tumor-bearing mice were injected subcutaneously with PBS or were co-injected with PBS and EC17 (1800 nmol/kg) on days 1, 2, 5, 7, 9, 12, 14, and 16 after tumor cell implantation. Animal gross morphology, behavior, and survival were monitored daily. As shown in FIG. 4, while the mice in the PBS control group all died at about 24-25 days after tumor implantation, the survival of mice treated with EC 17 was prolonged substantially.

### EXAMPLE 4

### Effect of Saponin-Enhanced EC17 Immunotherapy Alone (Without Cytokines) in Balb/c Mice Bearing a Seven-Day-Old Intraperitoneal M109 Tumor

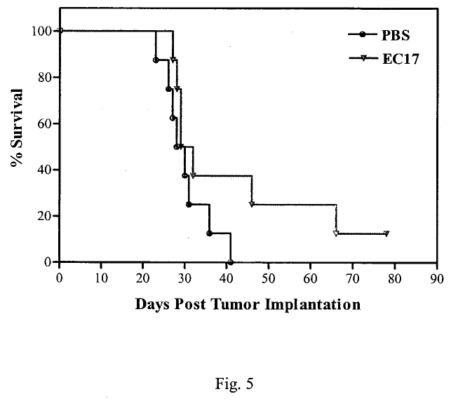

Six to eight week-old (˜20-22 grams) female Balb/c mice were immunized three times subcutaneously at one-week intervals with 35 μg of KLH-FITC formulated with 100 μg of GPI-0100. After confirming that anti-FITC antibody titers were high in all mice as described in Example 1, each animal was injected intraperitoneally with 0.5×10⁵ M109 cells. The cancer cells were then allowed to grow in vivo for 7 days. Thereafter, the tumor-bearing mice were injected intraperitoneally with PBS or with PBS and EC17 (1800 nmol/kg/day) on days 7-11, 14-18, and 21-25 after tumor cell implantation. EC17 and INF-α were dosed at 3 times per week. IL-2 was dosed at 5 times per week. Animal gross morphology, behavior, and survival were monitored daily. As shown in FIG. 5, EC17 alone exhibited a minor extension of lifespan of the tumor-bearing mice compared to the PBS control. Accordingly, the results shown in FIG. 4 and FIG. 5 taken together demonstrate that EC17 alone has significant antitumor effect at the early stage of tumor development. More importantly, the results shown in FIG. 3 and FIG. 5 taken together demonstrate that EC17 and cytokines, such as IL-2 and IFN-α, cause a synergistic increase in the lifespan of tumor-bearing mice compared to treatment with EC17 or cytokines alone.

### EXAMPLE 5

### Saponin Enhanced Immunotherapy (With Cytokines) Prevented Tumor Growth in Balb/c Mice Bearing a Subcutaneous M109 Tumor

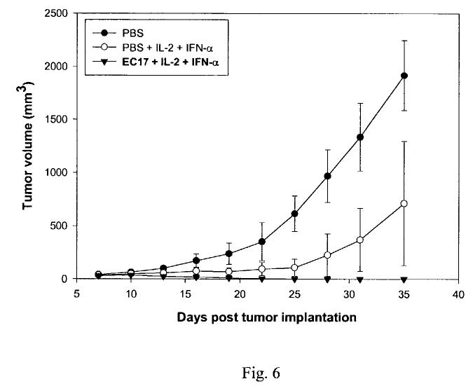

Six to eight week-old (˜20-22 grams) female Balb/c mice were immunized three times subcutaneously at one-week intervals with 35 μg of KLH-FITC formulated with 100 μg of GPI-0100. After confirming that anti-FITC antibody titers were high in all mice as described in Example 1, each animal was injected subcutaneously in the shoulder with 1×10⁶ M109 cells. The cancer cells were then allowed to grow for a week to 30-50 mm³. Thereafter, the tumor-bearing mice were injected intraperitoneally with PBS or were co-injected with PBS, IL-2 (40,000 IU/dose), and IFN-α (25,000 IU/dose), or with PBS, EC 17 (1800 nmol/kg), IL-2 (40,000 IU/dose), and IFN-α (25,000 IU/dose) on days 7-11, 14-18, and 21-25 after tumor cell implantation. EC17 and IL-2 were dosed at 5 times per week. IFN-α was dosed at 3 times per week. Tumor volumes were measured every other day using a caliper. As shown in FIG. 6, subcutaneous tumors in mice injected with EC17, IL-2, and IFN-α exhibited a decrease in size over 35 days post implantation compared to significant growth of tumors in mice injected with PBS or with PBS, IL-2, and IFN-α.

What is claimed is:

1. In a method of enhancing an endogenous immune response-mediated specific elimination of a population of pathogenic cells in a preimmunized host animal harboring the population wherein the members of the cell population have an accessible binding site for a ligand, and wherein the method comprises the step of administering to the host a composition comprising an immunogen or a hapten conjugated to the ligand wherein the immunogen or hapten is recognized by an endogenous antibody in the host or is recognized directly by an immune cell in the host, the improvement comprising the step of preimmunizing the host with the immunogen or an immunogenic hapten-carrier conjugate and a T_{H}1-biasing adjuvant to elicit a preexisting immunity.

2. The method of claim 1 further comprising the step of administering to the host at least one additional composition comprising a therapeutic factor wherein the factor is selected from the group consisting of a cell killing agent, a tumor penetration enhancer, a chemotherapeutic agent, an antimicrobial agent, a cytotoxic immune cell, and a compound capable of stimulating an endogenous immune response.

3. The method of claim 1 wherein the adjuvant is selected from the group consisting of an unmodified saponin adjuvant and a modified saponin adjuvant.

4. The method of claim 3 wherein the modified saponin adjuvant is lipid-modified.

5. The method of claim 1 wherein the adjuvant is a quillajasaponin adjuvant.

6. The method of claim 4 wherein the modified saponin adjuvant is a lipid-modified quillajasaponin adjuvant.

7. The method of claim 1 wherein the host is preimmunized with a composition comprising a hapten-carrier conjugate.

8. The method of claim 7 wherein the hapten is selected from the group consisting of fluorescein and dinitrophenyl.

9. A method of enhancing an immune response in a host animal harboring a population of pathogenic cells to eliminate said pathogenic cell population wherein the pathogenic cells have an accessible binding site for a ligand, said method comprising the steps of
administering to the host a T_{H}1-biasing adjuvant; and
administering to the host a composition comprising an immunogen conjugated to the ligand.

10. The method of claim 9 further comprising the step of administering to the host at least one additional composition comprising a therapeutic factor wherein the factor is selected from the group consisting of a cell killing agent, a tumor penetration enhancer, a chemotherapeutic agent, an antimicrobial agent, a cytotoxic immune cell, and a compound capable of stimulating an endogenous immune response.

11. The method of claim 9 wherein the adjuvant is selected from the group consisting of an unmodified saponin adjuvant and a modified saponin adjuvant.

12. The method of claim 11 wherein the modified saponin adjuvant is lipid-modified.

13. The method of claim 9 wherein the adjuvant is a quillajasaponin adjuvant.

14. The method of claim 12 wherein modified the saponin adjuvant is a lipid-modified quillajasaponin adjuvant.

15. A composition comprising therapeutically effective amounts of a T_{H}1-biasing adjuvant and a hapten-carrier conjugate wherein the hapten is selected from the group consisting of fluorescein and dinitrophenyl.

16. A composition comprising therapeutically effective amounts of a T_{H}1-biasing adjuvant and a ligand-immunogen conjugate.

17. A kit comprising a T_{H}1-biasing adjuvant and a hapten-carrier conjugate wherein the hapten is selected from the group consisting of fluorescein and dinitrophenyl.

18. A kit comprising a T_{H}1-biasing adjuvant, a hapten-carrier conjugate, and a ligand-hapten conjugate.

19. A kit comprising a T_{H}1-biasing adjuvant and a ligand-immunogen conjugate.

20. The kit of claim 19 wherein the immunogen is a hapten.

21. The kit of claim 20 wherein the hapten is selected from the group consisting of fluorescein or dintrophenyl.

22. The kit of claim 18 further comprising a therapeutic factor.

23. The kit of claim 22 wherein the therapeutic factor comprises a cytokine.

24. The kit of claim 19 further comprising a therapeutic factor.

25. The kit of claim 24 wherein the therapeutic factor comprises cytokine.

26. A kit comprising a T_{H}1-biasing adjuvant, an immunogen, and a ligand-immunogen conjugate.
